(19) United States
(12) Patent Application Publication
(10) Pub. No.: US 2005/0197304 A1
(43) Pub. Date: Sep. 8, 2005

(54) Nucleic acid therapy to enhance cartilage repair

(76) Inventor: Paul DiCesare, Irvington, NY (US)

Correspondence Address: KLAUBER & JACKSON, 411 HACKENSACK AVENUE, HACKENSACK, NJ, 07601, US

(21) Appl. No.: 10/886,947

(22) Filed: Jul. 8, 2004

Related U.S. Application Data
(60) Provisional application No. 60/485,669, filed on Jul. 8, 2003.

(51) Int. Cl. A61K 48/00; A61K 9/14

(52) U.S. Cl. 514/44; 424/486

(57) ABSTRACT

The present invention relates to the application of nucleic acid therapy for the repair and regeneration of cartilage. The invention encompasses the introduction of naked DNA encoding bioactive agent(s), whose expression stimulates and otherwise facilitates the repair and regeneration of cartilage. The present invention provides DNA constructs for introduction to the site of cartilage damage. Pharmaceutical compositions comprising nucleic acid encoding one or more bioactive factor, optionally with a matrix or polymer, are provided. Methods for expression of bioactive agent(s) are provided. Methods for enhancing cartilage repair and/or regeneration comprising introduction of bioactive factors as naked DNA are further provided. Methods for the treatment or prevention of cartilage damage in various orthopaedic and rheumatologic conditions are provided.

### CROSS-REFERENCE TO RELATED APPLICATIONS

The present application claims the benefit of priority pursuant to 35 U.S.C. § 119 to U.S. Provisional Application 60/485,669 filed Jul. 8, 2003, which is incorporated herein by reference in its entirety.

### FIELD OF THE INVENTION

The present invention relates generally to the application of nucleic acid therapy for the repair and regeneration of cartilage. The invention encompasses the introduction of naked DNA encoding bioactive agent(s), whose expression stimulates and otherwise facilitates the repair and regeneration of cartilage. Methods for the repair and/or regeneration of cartilage for treatment or prevention of cartilage damage in various orthopaedic and rheumatologic conditions are provided.

### BACKGROUND OF THE INVENTION

More than $215 billion is annually spent on treatment of musculoskeletal conditions in the United States [21]. Arthritis affects almost 1 out of every 8 Americans—about 32 million persons—with an annual price tag of about $82 billion. The impact of musculoskeletal conditions and injuries is expected to grow as the population both increases and ages in the coming decades [22]. Over the past decade the number of operations performed on knees has risen dramatically and currently account for almost half of all orthopaedic procedures, approximately 648,000 annually.

Arthritis, both rheumatoid and osteoarthritis, constitutes a major medical problem. In particular, degeneration of articular cartilage in osteoarthritis is a serious medical problem. Drugs are given to control the pain and to keep the swelling down, but the cartilage continues to be destroyed. Eventually, the joint must be replaced.

Whether articular cartilage is damaged from trauma or congenital anomalies, its successful clinical regeneration is poor at best. The inability of adult articular cartilage for self repair has been well recognized and has stimulated much interest. There are two major mechanisms of articular cartilage repair: intrinsic and extrinsic. Superficial or partial-thickness injuries that do not penetrate the subchondral bone rely on the intrinsic mechanism for repair. Soon after superficial injury, chondrocytes adjacent to the injured surfaces show a brief burst of mitotic activity associated with an increase in glycosaminoglycan and collagen synthesis. Despite these attempts at repair, there is no appreciable increase in the bulk of cartilage matrix and the repair process is rarely effective in healing the defects.

Osteochondral, or full-thickness, cartilage defects extend into the subchondral bone. Such defects arise after the detachment of osteochondritic dissecting flaps, fractured osteochondral fragments, or from chronic wear of degenerative articular cartilage. Osteochondral defects depend on the extrinsic mechanism for repair. Extrinsic healing relies on mesenchymal elements from subchondral bone to participate in the formation of new connective tissue. This fibrous tissue may or may not undergo metaplastic changes to form fibrocartilage. Even if fibrocartilage is formed, it does not display the same biochemical composition or mechanical properties of normal articular cartilage or subchondral bone and degenerates with use. The ensuing osteoarthritis may result in permanent disability and discomfort to the patient.

The response of normal articular cartilage to injury and damage often results in suboptimal repair because of the limited regenerative and reparative capabilities of chondrocytes [23, 24]. Current treatment options (e.g., drilling, microfracture, abrasion arthroplasty) are primarily aimed at stimulating extrinsic repair from cells deep to the tidemark. These techniques may ameliorate clinical symptoms in the short term but fail in the long term, since the resultant regenerate tissue, fibrocartilage, does not have the properties of hyaline cartilage.

A variety of tissue transplant grafts have also been advocated. Soft tissue grafts (perichondrial and periosteal grafts) have not gained widespread use due to the limited amounts of donor tissue and the tendency of this tissue to ossify. Autogenous osteochondral grafts (mosaicplasty) have shown promise in achieving repair; their drawbacks include donor site morbidity and the typically fibrocartilaginous character of the tissue at the graft-host interface. Autologous chondrocyte implantation offers the opportunity to regenerate hyaline-like cartilage in chondral defects [5, 25-27] [1, 2, 28, 29]. Among the limitations with this technique are that it requires at least two surgeries, is expensive, is limited to certain areas of the knee, is indicated primarily for younger patients (under 45 years), requires monitoring for safety, viability, and microbial integrity of the autologous cells while they are in culture over a 4- to 5-week period prior to implantation, and carries unknown long-term donor site morbidity.

Cartilage has been grown by seeding synthetic polymeric matrices with dissociated cells, which are then implanted to form new cartilage (as described in U.S. Pat. Nos. 5,041,138 and 4,846,835). Cartilage has also been grown from an injected or implanted ionically crosslinked hydrogel-chondrocyte suspension (Atala, et al. (1993) J. Urology 150(2-part 2): 745-747). Injection of dissociated chondrocytes directly into a defect has also been described as a means for forming new cartilage (Brittberg, et al. (1994) N. E. J. Med. 331:889-895). Cartilage was harvested from minor load-bearing regions on the upper medial femoral condyle of the damaged knee, cultured, and implanted two to three weeks after harvesting. Freed and Grande cultured mature chondrocytes from New Zealand white rabbits in vitro onto polyglycolic acid (PGA) scaffolds and imbedded the PGA-cells into a full thickness articular cartilage defect in syngeneic New Zealand white rabbits (Freed and Grande (1994) J. Biomed. Mater. Res. 28:891). The PGA-cells implant showed normal articular cartilage histology, but did not have normal subchondral bone.

A disadvantage of these systems is that the chondrocytes must be obtained from the patient, typically by a biopsy, cultured, and then implanted on the matrix. This is relatively easy in laboratory animals, but presents greater logistical problems in humans where a defect is created by the biopsy required to provide cells for repair of another defect. Moreover, if the defect includes a part of the underlying bone, this is not corrected using chondrocytes, which are already differentiated and will not form new bone. The bone is required to support the new cartilage.

Stem cells are cells which are not terminally differentiated and can divide without limit to yield cells that are either stem cells or which irreversibly differentiate to yield a new type of cell. Chondro/osteoprogenitor cells, which are bipotent with the ability to differentiate into cartilage or bone, have been isolated from bone marrow (Owen, J. (1988) Cell Sci. Suppl. 10, 63-76; and U.S. Pat. No. 5,226,914 to Caplan, et al.). Mesenchymal stem cells, which are capable of differentiating into chondrocytes (cartilage), osteoblasts (bone), myotubes (muscle), adipocytes (fat), and connective tissue cells, have been isolated and their use described for cartilage regeneration and repair (U.S. Pat. No. 5,827,735).

Several peptide growth and differentiation factors have been identified that appear to control cellular events associated with cartilage formation and repair. The bone morphogenetic proteins (BMPs) comprise a family of soluble extracellular proteins that control osteogenic cell fate, initiate chondrogenesis, and maintain the chondrocyte phenotype. Fetal osteoblasts in vitro and in vivo normally express BMP genes [30], and recombinant BMP proteins initiate cartilage and bone progenitor cell differentiation [31, 32]. BMP-2 is a potent stimulator of articular cartilage proteoglycan synthesis in vitro and in vivo [33, 34]. BMP-3 and BMP-4 prevent chondrocyte dedifferentiation and maintain the differentiated phenotype in vitro [35].

Growth and differentiation factors have been used to facilitate articular cartilage repair [36-38]. Relatively large doses (microgram amounts) are required to stimulate new cartilage formation in animals, however, raising the concern that future human therapies will be expensive and may possess an increased risk of toxicity. As an alternative, regional gene therapy can be more cost-effective, because production of DNA is much less expensive than traditional methods of protein production [39]. Gene therapy shows promise as a more efficient way to deliver chondrogenic factors in vivo since the cell-mediated synthesis and delivery are likely associated with efficient targeting to cell surface receptors; consequently, less protein will be required to achieve a clinically desirable effect [40-42]. Local delivery of cDNAs encoding several potentially antiarthritic genes has been achieved by ex vivo transfer with an adenovirus or retrovirus [43-48].

Gene therapy for arthritis has focused on the use of viral vectors to introduce the desired gene, typically one that encodes an inhibitor of cartilage degeneration (e.g., members of the interleukin family) into synoviocytes or chondrocytes ex vivo. The transfected cells are then implanted with the expectation that they will express the desired gene and prevent further deterioration [49]. Concerns have been voiced regarding the use of retroviruses, however, including the risk of insertional mutagenesis, the difficulty in targeting specific tissues, and immunogenicity to viral particles.

Damaged articular cartilage lacks the ability to restore a normal, hyaline joint surface; if left untreated, such damage often results in arthritic degeneration. Conventional therapeutic options have not resulted in the regeneration of normal cartilage [1, 2]. Another approach to stimulating regeneration of cartilage in articular defects involves the use of peptide growth factors. These factors are expensive, typically require large therapeutic dosage, may be degraded, may diffuse away from the desired location, and/or may require activation of a latent form. Therefore, in view of the aforementioned deficiencies attendant with these prior art methods and therapies, it should be apparent that there still exists a need in the art for a less costly, efficient and effective methods and therapies for the repair and regeneration of cartilage.

The citation of references herein shall not be construed as an admission that such is prior art to the present invention.

### SUMMARY OF THE INVENTION

The present invention provides a gene delivery system to facilitate cartilage repair. The present invention provides regional genetically engineered constructs expressing a bioactive agent(s) for stimulating and otherwise facilitating the repair and regeneration of cartilage. The invention described herein is distinct from previous techniques in this field which have employed various vectors, primarily viral, that has given rise to such concerns as insertional mutagenesis, difficulty in targeting specific tissues, and host immunogenicity to viral antigens.

In accordance with the present invention, early repair cells that populate a full-thickness articular cartilage defect incorporate implanted naked plasmid DNA capable of expressing a bioactive agent(s) and, once transfected, will serve as local bioreactors, transiently producing the bioactive agent(s), including for instance peptide factors, that will stimulate hyaline-like articular cartilage repair.

In accordance with the present invention, a method is provided for producing cartilage at a cartilage defect site in vivo comprising administering to the defect site naked DNA encoding one or more bioactive agent capable of stimulating the production of cartilage.

In a further aspect, the invention provides a method for enhancing cartilage repair in vivo comprising administering to a location where cartilage repair is desired naked DNA encoding one or more bioactive agent capable of enhancing cartilage repair.

In an additional aspect, the invention provides a method for expressing a bioactive agent in chondrocytes in vivo comprising administering naked DNA encoding one or more bioactive agent to a region in vivo where chondrocytes or chondrocyte progenitors are located such that the chondrocytes or chondrocyte progenitors take up the naked DNA and express the bioactive agent(s) encoded thereby.

In one embodiment, the naked DNA encodes a bioactive agent selected from the group of cartilage morphogens and factors or peptides which block inhibitory signals preventing the repair or regeneration of cartilage. In a particular embodiment, the agent is a factor selected from the group of cellular growth factors, factors that stimulate chondrogenesis, factors that stimulate migration of stromal cells and factors that stimulate matrix deposition. In a particularly preferred embodiment, the agent is selected from the group of an FGF, a TGF, and a BMP. In an exemplary embodiment, the agent is BMP-2.

The naked DNA may contain or the bioactive agent may be expressed to include a label, unrelated peptide or indicator, for instance an unrelated peptide, an antigenic site, a compound or peptide conferring antibiotic resistance or sensitivity, an enzyme, and/or a radioactive element which can be utilized as an indicator of the presence of the naked DNA and/or the expression of the biactive agent(s) in a cellular sample or in vivo. The presence or amount of the label, unrelated peptide or indicator may be examined by known techniques, which may vary with the nature of the label attached. In the instance where a radioactive label, such as the isotopes ³H, ¹⁴C, ³²P, ³⁵S, ³⁶Cl, ⁵¹Cr, ⁵⁷Co, ⁵⁸ Co, ⁵⁹Fe, ⁹⁰Y, ¹²⁵I, ¹³¹I, and ¹⁸⁶Re are used, known currently available counting procedures may be utilized. In the instance where the label is an enzyme, detection may be accomplished by any of the presently utilized calorimetric, spectrophotometric, fluorospectrophotometric, amperometric or gasometric techniques known in the art.

Alternatively, or in addition, the naked DNA may contain or the bioactive agent may be expressed to include a control element or transcription element or binding site, etc., whereby the expression of the encoded agent(s) or other unrelated peptide can be modulated or otherwise controlled, i.e. activated or inactivated. Such a construct provides an inducible or extinguishable system, whereby the enhancement of or extent of cartilage regeneration or repair can be modulated, turned on or turned off in certain circumstances. Such a system may be constructed to respond to the addition of a factor or chemical agent, for instance, which can be administered or added.

In a further embodiment, the present invention relates to certain therapeutic methods for the repair and/or regeneration of cartilage based on the expression of one or more bioactive agent capable of stimulating or otherwise enhancing cartilage regeneration and/or repair.

More specifically, the therapeutic method generally referred to herein could include the method for the treatment of various orthopedic and rheumatologic conditions including degenerative connective tissue diseases or in the event of physical trauma. In particular, the naked DNA encoding bioactive agent(s), could be prepared in pharmaceutical formulations for administration in instances wherein cartilage repair and/or regeneration is appropriate, such as to treat fracture nonunion, large segmental bone or cartilage defects, osteoarthritis, collagen disorders, dwarfism including camptomelic dysplasia, pseudoachondroplasia, multiuple epiphyseal dysplasia.

Other objects and advantages will become apparent to those skilled in the art from a review of the following description which proceeds with reference to the following illustrative drawings.

### BRIEF DESCRIPTION OF THE DRAWINGS

FIG. 1. Histological appearance of repair tissue at 6 weeks postoperatively. Safranin O staining; original magnification ×64. Left: Untreated implant; Right: Implant containing rhBMP-2.

FIG. 2. Left: Standard curve for the PCR quantitation. Right: Densitometer quantitation of diffusion kinetics from polystyrene slab-sponge construct.

FIG. 3. Histology of defect implanted with marker plasmid for β-gal at 1 wk. Left: Low-power, Hematoxylin and eosin (H&E) staining. Middle: Low-power, immunostaining for β-gal. Right: High-power, immunostaining for β-gal.

FIG. 4. Left: Standard curve for the effect of purified BMP-2 protein on the activity of alkaline phosphatase in W-20-17 cells. Right: Activity of recombinant BMP-2 in conditioned medium expressed from 293T cells.

FIG. 5 depicts the results of a rabbit defect study comparing repair scores in vivo of Naked BMP-2-Gene-Treated, plasmid treated (control) and BMP-2 protein treated rabbits.

FIG. 6 depicts the nucleic acid sequence of human bone morphogenetic protein (BMP-2) (SEQ ID NO: 1).

FIG. 7 depicts the amino acid sequence of human bone morphogenetic protein (BMP-2) (SEQ ID NO: 2).

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Therefore, if appearing herein, the following terms shall have the definitions set out below.

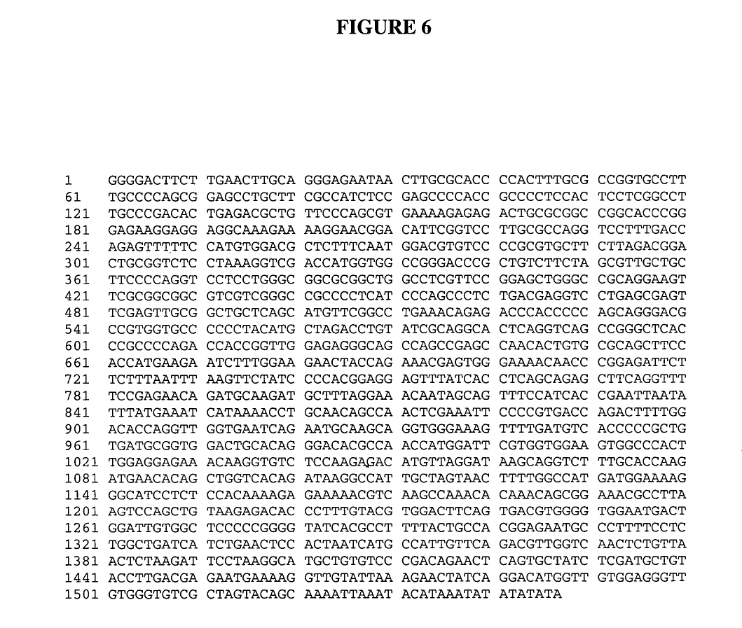

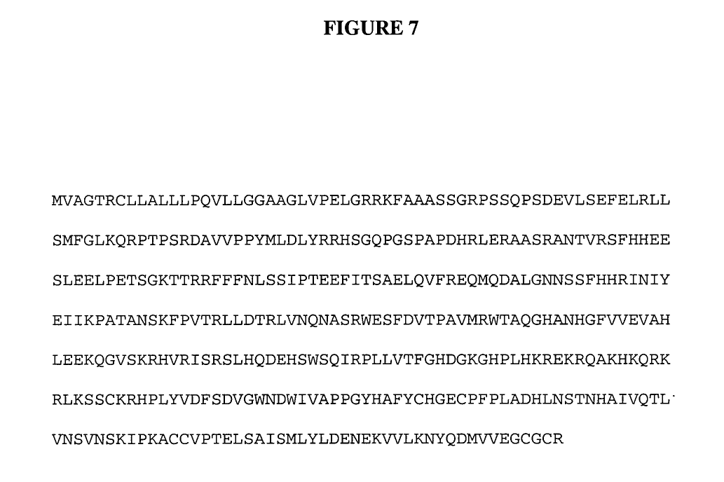

The terms “bioactive agent”, “bioactive agent(s)”, “bioactive factors” and any variants not specifically listed, may be used herein interchangeably, and as used throughout the present application and claims refer to peptides, compounds or factors which are capable of enhancing or otherwise stimulating the generation, production, regeneration and/or repair of cartilage and will include cartilage morphogens and factors or peptides which block inhibitory signals preventing the repair or regeneration of cartilage. Examples of bioactive factors of use in the present invention include, but are not limited to cellular growth factors (for instance TGF-β), factors that stimulate chondrogenesis (for example bone morphogenetic proteins (BMPs) that promote cartilage formation), factors that stimulate migration of stromal cells, factors that stimulate matrix deposition and osteogenic molecules. Bioactive agent(s) further include anti-inflammatories and immunosuppressants. Bioactive agent(s) include proteins which ordinarily form part of the cartilage extracellular matrix, including for example, collagens, elastic fibers, reticular fibers, glycoproteins or glycosaminoglycans (heparin sulfate, chondroitin-4-sulfate, chondroitin-6-sulfate, dermatan sulfate, keratin sulfate), and hyaluronic acid. Factors for use in the invention include FGF, TGFβ, BMPs (including BMP-2, BMP-2B, BMP-7, BMP-11), cartilage oligomeric matrix protein (COMP), IL-10, IL-1 receptor antagonist, latent TGFβ-binding protein (LTBP-3), Smads and GDF-5. Exemplary of a bioactive agent for use in the present invention is BMP-2 as described and demonstrated in the Examples herein, in one embodiment such BMP-2 having the nucleic acid and amino acid sequences as described herein and presented in FIGS. 6 and 7, respectively (SEQ ID NOS: 1 and 2, respectively). The present invention contemplates that bioactive agent(s) include combinations of one or more bioactive agent, particularly and including wherein their activities or capabilities may be complementary or additive. Accordingly, proteins displaying substantially equivalent or altered activity are likewise contemplated. These modifications may be deliberate, for example, such as modifications obtained through site-directed mutagenesis, or may be accidental, such as those obtained through mutations in hosts that are producers of the complex or its named subunits. Also, the terms “bioactive agent”, “bioactive agent(s)”, “bioactive factors” and the exemplary “BMP-2”are intended to include within their scope proteins specifically recited herein as well as all substantially homologous analogs and allelic variations.

The term “cartilage” as used herein is generally recognized in the art, and refers to a specialized type of dense connective tissue comprising cells embedded in an extracellular matrix (ECM) (see, for example, Cormack, 1987, Ham's Histology, 9th Ed., J. B. Lippincott Co., pp. 266-272). The biochemical composition of cartilage differs according to type, however, the general composition of cartilage comprises chondrocytes surrounded by a dense extracellular matrix (ECM) consisting of collagen, proteoglycans and water. Several types of cartilage are recognized in the art, including, for example, hyaline or articular cartilage such as that found within the joints, fibrous cartilage such as that found within the meniscus and costal regions, and elastic cartilage. The production, repair and/or regeneration of any type of cartilage is intended to fall within the scope of the invention.

The amino acid residues described herein are preferred to be in the “L” isomeric form. However, residues in the “D” isomeric form can be substituted for any L-amino acid residue, as long as the desired fuctional property of immunoglobulin-binding is retained by the polypeptide. NH₂ refers to the free amino group present at the amino terminus of a polypeptide. COOH refers to the free carboxy group present at the carboxy terminus of a polypeptide. In keeping with standard polypeptide nomenclature, J. Biol. Chem., 243:3552-59 (1969), abbreviations for amino acid residues are shown in the following Table of Correspondence:

TABLE OF CORRESPONDENCE

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| W | Trp | tryptophan |
| R | Arg | arginine |
| D | Asp | aspartic acid |
| N | Asn | asparagine |
| C | Cys | cysteine |

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues. The above Table is presented to correlate the three-letter and one-letter notations which may appear alternately herein.

A “replicon” is any genetic element (e.g., plasmid, chromosome, virus) that functions as an autonomous unit of DNA replication in vivo; i.e., capable of replication under its own control.

A “vector” is a replicon, such as plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence. This sequence encodes a signal peptide, N-terminal to the polypeptide, that communicates to the host cell to direct the polypeptide to the cell surface or secrete the polypeptide into the media, Signal sequences can be found associated with a variety of proteins native to prokaryotes and eukaryotes.

The term “oligonucleotide” is defined as a molecule comprised of two or more ribonucleotides, preferably more than three. Its exact size will depend upon many factors which, in turn, depend upon the ultimate function and use of the oligonucleotide.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

As used herein, the terms “restriction endonucleases” and “restriction enzymes” refer to bacterial enzymes, each of which cut double-stranded DNA at or near a specific nucleotide sequence.

A cell has been “transformed” by exogenous or heterologous DNA when such DNA has been introduced inside the cell. The transforming DNA may or may not be integrated (covalently linked) into chromosomal DNA making up the genome of the cell.

In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra. It should be appreciated that also within the scope of the present invention are DNA sequences encoding a bioactive agent(s) which code for a bioactive agent(s) having the same nucleic acid sequence as known or as that disclosed herein (for instance, SEQ ID NO:), but which are degenerate to said sequence (for instance SEQ ID NO:). By “degenerate to” is meant that a different three-letter codon is used to specify a particular amino acid. It is well known in the art that the following codons can be used interchangeably to code for each specific amino acid:

| | | |
|---|---|---|
| Phenylalanine | (Phe or F) | UUU or UUC |
| Leucine | (Leu or L) | UUA or UUG or CUU or CUC or CUA or CUG |
| Isoleucine | (Ile or I) | AUU or AUC or AUA |
| Methionine | (Met or M) | AUG |
| Valine | (Val or V) | GUU or GUC or GUA or GUG |
| Serine | (Ser or S) | UCU or UCC or UCA or UCG or AGU or AGC |
| Proline | (Pro or P) | CCU or CCC or CCA or CCG |
| Threonine | (Thr or T) | ACU or ACC or ACA or ACG |
| Alanine | (Ala or A) | GCU or GCG or GCA or GCG |
| Tyrosine | (Tyr or Y) | UAU or UAC |
| Histidine | (His or H) | CAU or CAC |
| Glutamine | (Gln or Q) | CAA or CAG |
| Asparagine | (Asn or N) | AAU or AAC |
| Lysine | (Lys or K) | AAA or AAG |
| Aspartic Acid | (Asp or D) | GAU or GAC |
| Glutamic Acid | (Glu or E) | GAA or GAG |
| Cysteine | (Cys or C) | UGU or UGC |
| Arginine | (Arg or R) | CGU or CGC or CGA or CGG or AGA or AGG |
| Glycine | (Gly or G) | GGU or GGC or GGA or GGG |
| Tryptophan | (Trp or W) | UGG |
| Termination codon | | UAA (ochre) or UAG (amber) or UGA (opal) |

It should be understood that the codons specified above are for RNA sequences. The corresponding codons for DNA have a T substituted for U.

Mutations can be made in the bioactive agent(s) for use in the present invention (including for instance SEQ ID NO:) such that a particular codon is changed to a codon which codes for a different amino acid. Such a mutation is generally made by making the fewest nucleotide changes possible. A substitution mutation of this sort can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present invention should be considered to include seguences containing conservative changes which do not significantly alter the activity or binding characteristics of the resulting protein.

The following is one example of various groupings of amino acids:

Amino acids with nonpolar R groups:
Alanine, Valine, Leucine, Isoleucine, Proline, Phenylalanine, Tryptophan, Methionine

Amino acids with uncharged polar R groups:
Glycine, Serine, Threonine, Cysteine, Tyrosine, Asparagine, Glutamine

Amino acids with charged polar R groups (negatively charged at Ph 6.0):
Aspartic acid, Glutamic acid

Basic amino acids (positively charged at pH 6.0):
Lysine, Arginine, Histidine (at pH 6.0)

Another grouping may be those amino acids with phenyl groups:
Phenylalanine, Tryptophan, Tyrosine

Another grouping may be according to molecular weight (i.e., size of R groups):

| | |
|---|---|
| Glycine | 75 |
| Alanine | 89 |
| Serine | 105 |
| Proline | 115 |
| Valine | 117 |
| Threonine | 119 |
| Cysteine | 121 |
| Leucine | 131 |
| Isoleucine | 131 |
| Asparagine | 132 |
| Aspartic acid | 133 |
| Glutamine | 146 |
| Lysine | 146 |
| Glutamic acid | 147 |
| Methionine | 149 |
| Histidine (at pH 6.0) | 155 |
| Phenylalanine | 165 |
| Arginine | 174 |
| Tyrosine | 181 |
| Tryptophan | 204 |

Particularly preferred substitutions are:
Lys for Arg and vice versa such that a positive charge may be maintained;
Glu for Asp and vice versa such that a negative charge may be maintained;
Ser for Thr such that a free —OH can be maintained; and
   Gln for Asn such that a free NH₂ can be maintained.

Amino acid substitutions may also be introduced to substitute an amino acid with a particularly preferable property. For example, a Cys may be introduced a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces—turns in the protein's structure.

Two amino acid sequences are “substantially homologous” when at least about 70% of the amino acid residues (preferably at least about 80%, and most preferably at least about 90 or 95%) are identical, or represent conservative substitutions.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

An “antibody” is any immunoglobulin, including antibodies and fragments thereof, that binds a specific epitope. The term encompasses polyclonal, monoclonal, and chimeric antibodies, the last mentioned described in further detail in U.S. Pat. Nos. 4,816,397 and 4,816,567. Exemplary antibody molecules are intact immunoglobulin molecules, substantially intact immunoglobulin molecules and those portions of an immunoglobulin molecule that contains the paratope, including those portions known in the art as Fab, Fab′, F(ab′)₂ and F(v).

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may therefore contain an antibody molecule having a plurality of antibody combining sites, each immunospecific for a different antigen; e.g., a bispecific (chimeric) monoclonal antibody.

The phrase “pharmaceutically acceptable” refers to molecular entities and compositions that are physiologically tolerable and do not typically produce an allergic or similar untoward reaction, such as gastric upset, dizziness and the like, when administered to a human.

The phrase “therapeutically effective amount” is used herein to mean an amount sufficient to stimulate or otherwise enhance, and preferably increase by at least about 30 percent, more preferably by at least 50 percent, most preferably by at least 90 percent, a clinically significant increase in the repair and/or regeneration of cartilage, including, for instance, an increase in the differentiation of cells into chondrocytes, the proliferation of chondrocytes, the expression of collagens (for instance type II, IX, XI), the expression of the proteoglycan aggrecan, and/or the expression of the noncollagenous protein cartilage oligomeric matrix protein(COMP).

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

The term “standard hybridization conditions” refers to salt and temperature conditions substantially equivalent to 5×SSC and 65° C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20^{N}C below the predicted or determined Tₘ with washes of higher stringency, if desired.

The present invention provides a gene delivery system to facilitate cartilage repair. The present invention provides regional genetically engineered constructs expressing a bioactive agent(s) for stimulating and otherwise facilitating the repair and regeneration of cartilage. The invention described herein is distinct from previous techniques in this field which have employed various vectors, primarily viral, that has given rise to such concerns as insertional mutagenesis, difficulty in targeting specific tissues, and host immunogenicity to viral antigens.

In accordance with the present invention, early repair cells that populate a full-thickness articular cartilage defect incorporate implanted naked plasmid DNA capable of expressing a bioactive agent(s) and, once transfected, will serve as local bioreactors, transiently producing the bioactive agent(s), including for instance peptide factors, that will stimulate hyaline-like articular cartilage repair.

In accordance with the present invention, a method is provided for producing cartilage at a cartilage defect site in vivo comprising administering to the defect site naked DNA encoding one or more bioactive agent capable of stimulating the production of cartilage.

In a further aspect, the invention provides a method for enhancing cartilage repair in vivo comprising administering to a location where cartilage repair is desired naked DNA encoding one or more bioactive agent capable of enhancing cartilage repair. In an additional aspect, the invention provides a method for expressing a bioactive agent in chondrocytes in vivo comprising administering naked DNA encoding one or more bioactive agent to a region in vivo where chondrocytes or chondrocyte progenitors are located such that the chondrocytes or chondrocyte progenitors take up the naked DNA and express the bioactive agent(s) encoded thereby.

In one embodiment, the naked DNA encodes a bioactive agent selected from the group of cartilage morphogens and factors or peptides which block inhibitory signals preventing the repair or regeneration of cartilage. In a particular embodiment, the agent is a factor selected from the group of cellular growth factors, factors that stimulate chondrogenesis, factors that stimulate migration of stromal cells and factors that stimulate matrix deposition. In a particularly preferred embodiment, the agent is selected from the group of an FGF, a TGF, and a BMP. In an exemplary embodiment, the agent is BMP-2.

As stated above, the present invention also relates to a recombinant DNA molecule or cloned gene, or a degenerate variant thereof, which encodes a bioactive agent(s) or a fragment thereof. In one embodiment of the present invention, recombinant DNA molecule or cloned gene is provided which encodes BMP-2, having an amino acid sequence set forth in FIG. 7 (SEQ ID NO: 2). Preferably, a nucleic acid molecule, in particular a recombinant DNA molecule or cloned gene, encoding the BMP-2 has a nucleotide sequence or is complementary to a DNA sequence shown in FIG. 6 (SEQ ID NO: 1).

The genetically engineered constructs or naked DNA for use in the invention may be prepared in pharmaceutical compositions, with a suitable carrier and at a strength effective for administration by various means to a patient experiencing a cartilage defect for the treatment thereof. A variety of administrative techniques may be utilized, among them parenteral techniques such as subcutaneous, intravenous and intraperitoneal injections, catheterizations and the like. Average quantities of the constructs or naked DNA may vary and in particular should be based upon the recommendations and prescription of a qualified physician or veterinarian.

The present invention further contemplates therapeutic compositions useful in practicing the therapeutic methods of this invention. A therapeutic composition includes, in admixture, a pharmaceutically acceptable excipient (carrier) and one or more of a bioactive agent(s) encoded by naked DNA, as described herein as an active ingredient. In a preferred embodiment, the composition comprises a naked DNA construct encoding a cartilage morphogen. In a particular embodiment the composition comprises a naked DNA construct encoding BMP-2.

The preparation of therapeutic compositions which contain nucleic acids as active ingredients is well understood in the art. Such compositions are prepared as liquid solutions or suspensions (e.g. for injection), however, solid forms suitable for solution in, or suspension in, liquid prior to injection can also be prepared. The preparation can also be emulsified. The active therapeutic ingredient can be mixed with excipients which are pharmaceutically acceptable and compatible with the active ingredient. Suitable excipients are, for example, water, saline, EDTA, EGTA, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

The DNA or nucleic acid can be formulated into the therapeutic composition as neutralized pharmaceutically acceptable salt forms. Pharmaceutically acceptable salts include the acid addition and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

In one embodiment, the therapeutic bioactive agent(s) encoding compositions are conventionally administered intravenously, as by injection of a unit dose, for example. In a preferred embodiment, the therapeutic bioactive agent(s) encoding compositions are injected In a further preferred embodiment, the therapeutic bioactive agent(s) encoding compositions are administered directly at the site of cartilage damage or injury, for instance by surgically pressing the composition, including a composition of the bioactive agent encoding nucleic acid in a carrier matrix or scaffold, including but not limited to a collagen sponge. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered depends on the subject to be treated, capacity of the subject's immune system to utilize the active ingredient, the extent of cartilage damage or size of cartilage defect, and degree of cartilage repair or regeneration desired. Precise amounts of active ingredient required to be administered depend on the judgment of the practitioner and are peculiar to each individual. Suitable dosages would also depend on the route of administration. AS exemplified herein, 70 ug of naked DNA encoding the bioactive agent BMP-2 is administered directly for a 3 mm defect. Certainly, the dosage would increase for size of defect and may also be increased based on the size or weight of the mammal or individual being treated. Suitable final dosages of bioactive agent or protein may range from about 0.1 to 20, preferably about 0.5 to about 10, and more preferably one to several, milligrams of active ingredient per kilogram body weight of individual per day and depend on the route of administration.

The therapeutic compositions may further include an effective amount of the bioactive agent, and one or more of the following active ingredients: an anti-inflammatory, an anti-arthritic, a steroid.

The naked DNA of the present invention encoding one or more bioactive agent(s) may be combined for implantation or administration with other molecules, including other bioactive agents as proteins or factors or extracellular matrix proteins (for example osteogenic molecules (osteogenic protein-1, BMP-7), aggregan) or may be combined with or embedded in polymeric carrier(s), biodegradable or biomimetic matrices or in a scaffold.

The carrier, matrix or scaffold may be of any material that will allow the naked DNA composition to be incorporated and expressed and will be compatible with the addition of cells or in the presence of cells. Preferably, the carrier matrix or scaffold is predominantly non-immunogenic, does not induce a significant inflammatory response and is biodegradable. Examples of biodegradable materials which may be used include polyglycolic acid (PGA), polylactic acid (PLA), hyaluronic acid, catgut suture material, gelatin, cellulose, nitrocellulose, collagen, albumin, fibrin, alginate, cotton, or other naturally-occurring biodegradable materials. It may be preferable to sterilize the matrix or scaffold material prior to implantation, e.g., by treatment with ethylene oxide or by gamma irradiation or irradiation with an electron beam. In addition, a number of other materials may be used to form the scaffold or framework structure, including but not limited to: nylon (polyamides), dacron (polyesters), polystyrene, polypropylene, polyacrylates, polyvinyl compounds (e.g., polyvinylchloride), polycarbonate (PVC), polytetrafluorethylene (PTFE, teflon), thermanox (TPX), polymers of hydroxy acids such as polylactic acid (PLA), polyglycolic acid (PGA), and polylactic acid-glycolic acid (PLGA), polyorthoesters, polyanhydrides, polyphosphazenes, and a variety of polyhydroxyalkanoates, and combinations thereof.

Matrices suitable include a polymeric mesh or sponge and a polymeric hydrogel. In the preferred embodiment, the matrix is biodegradable over a time period of less than a year, more preferably less than six months, most preferably over two to ten weeks. In the case of joint surface application, the degradation period is typically about twelve to twenty-four weeks. In the case where weight bearing or high shear stress is not an issue, the degradation period is typically about five to ten weeks. The term bioerodible or biodegradable, as used herein, means a polymer that dissolves or degrades within a period that is acceptable in the desired application, less than about six months and most preferably less than about twelve weeks, once exposed to a physiological solution of pH 6-8 having a temperature of between about 25° C. and 38° C. The polymer composition, as well as method of manufacture, can be used to determine the rate of degradation. For example, mixing increasing amounts of polylactic acid with polyglycolic acid decreases the degradation time.

In a preferred embodiment, the polymers form fibers which are intertwined, woven, or meshed to form a matrix having an interstitial spacing of between 100 and 300 microns. Meshes of polyglycolic acid that can be used can be obtained commercially from surgical supply companies (e.g., Ethicon, N.J.). Sponges can also be used. As used herein, the term “fibrous” refers to either a intertwined, woven or meshed matrix or a sponge matrix. The matrix is preferably shaped to fill the defect. In most cases this can be achieved by trimming the polymer fibers with scissors or a knife; alternatively, the matrix can be cast from a polymer solution formed by heating or dissolution in a volatile solvent. A hydrogel is defined as a substance formed when an organic polymer (natural or synthetic) is cross-linked via covalent, ionic, or hydrogen bonds to create a three-dimensional open-lattice structure which entraps water molecules to form a gel. In general, these polymers are at least partially soluble in aqueous solutions, such as water, buffered salt solutions, or aqueous alcohol solutions, that have charged side groups, or a monovalent ionic salt thereof.

In addition and in a further embodiment, the naked DNA of the present invention encoding one or more bioactive agent(s) may be combined for implantation or administration with cells, including chondrocytes, stromal cells, or stem cells, particularly mesenchymal stem cells or cells capable of differentiating along the mesenchymal lineage. Cartilage has been grown by seeding synthetic polymeric matrices with dissociated cells, which are then implanted to form new cartilage (as described in U.S. Pat. Nos. 5,041,138 and 4,846,835). Chondro/osteoprogenitor cells, which are bipotent with the ability to differentiate into cartilage or bone, have been isolated from bone marrow (Owen, J. (1988) Cell Sci. Suppl. 10, 63-76; and U.S. Pat. No. 5,226,914 to Caplan, et al.). Mesenchymal stem cells, which are capable of differentiating into chondrocytes (cartilage), osteoblasts (bone), myotubes (muscle), adipocytes (fat), and connective tissue cells, have been isolated and their use described for cartilage regeneration and repair (U.S. Pat. No. 5,827,735). The implantation of a biocompatible scaffold and cells for attachment to the scaffold for cartilage repair is described by Naughton in U.S. Pat. No. 5,842,477. In any such case, the cells may be implanted in combination with the naked DNA of the present invention or may first be transformed with the naked DNA of the present invention and then implanted at the cartilage defect site.

In one embodiment cells, including chondrocytes, chondrocyte progenitors or mesenchymal stem cells, are seeded onto the matrix by application of a cell suspension to the matrix. This can be accomplished by soaking the matrix in a cell culture container, or injection or other direct application of the cells to the matrix. Media should be washed from the cells and matrix prior to implantation.

The matrix seeded with the naked DNA, with or without cells, is implanted at the site of the defect using standard surgical techniques. The matrix can be seeded and cultured in vitro prior to implantation, seeded and immediately implanted, or implanted and then seeded with cells. In the embodiment using cells, cells are seeded onto and into the matrix and may be cultured in vitro for between approximately sixteen hours and two weeks. It is only critical that the cells be attached to the matrix. Cell density at the time of seeding or implantation should be approximately 25,000 cells/mm.sup.3.

In an embodiment utilizing hydrogel, the naked DNA (with or without cells) is mixed with the hydrogel solution and injected directly into a repair site, prior to hardening of the hydrogel. However, the matrix may also be molded and implanted in one or more different areas of the body to suit a particular application. This application is particularly relevant where a specific structural design is desired or where the area into which the cells are to be implanted lacks specific structure or support. One could also apply an external mold to shape the injected solution. Additionally, by controlling the rate of polymerization, it is possible to mold the cell-hydrogel injected implant like one would mold clay. Alternatively, the mixture can be injected into a mold, the hydrogel allowed to harden, then the material implanted.

The composition of the invention can be injected via a syringe and needle directly into a specific area wherever a bulking agent is desired, especially soft tissue defects. The suspension can also be injected as a bulking agent for hard tissue defects, such as bone or cartilage defects, either congenital or acquired disease states, or secondary to trauma, burns, or the like. The injection in these instances can be made directly into the needed area with the use of a needle and syringe under local or general anesthesia. The composition can be injected percutaneously by direct palpation. Alternatively, the composition can be injected through a catheter or needle with fluoroscopic, sonographic, computer tomography, magnetic resonance imaging or other type of radiologic guidance.

In one embodiment, the cartilage defect site into which the construct(s) of the present invention will be placed is treated, preferably prior to implantation, to degrade the pre-existing cartilage at the defect site, freeing cells to migrate into the scaffold of the implant and promoting the orderly deposition of new cartilage. Methods of such treatment include enzymatic treatment, abrasion or microdrilling. According to a further embodiment, the preparation of naked DNA of the invention can be injected or implanted into the degraded cartilage at the defect site, e.g., into the surrounding cells or into the walls of the defect, providing a source of biological factors that induce migration of stromal cells from the degraded cartilage to the implant.

In a further embodiment, the present invention relates to certain therapeutic methods for the repair and/or regeneration of cartilage based on the expression of one or more bioactive agent capable of stimulating or otherwise enhancing cartilage regeneration and/or repair.

More specifically, the therapeutic method generally referred to herein could include the method for the treatment of various orthopedic and rheumatologic conditions including degenerative connective tissue diseases or in the event of physical trauma. In particular, the naked DNA encoding bioactive agent(s), could be prepared in pharmaceutical formulations for administration in instances wherein cartilage repair and/or regeneration is appropriate, such as to treat fracture nonunion, large segmental bone or cartilage defects, ostoarthritis, collagen disorders, dwarfism including camptomelic dysplasia, pseudoachondroplasia, multiuple epiphyseal dysplasia.

The possibilities both diagnostic and therapeutic that are raised by the introduction of naked DNA expressing a bioactive agent(s) to a site of cartilage damage or trauma derive from the fact that the cells at the site incorporate and express the bioactive agent(s) and the local expression of the agent(s) facilitates the regeneration of cartilage and repair of a defect. As suggested earlier and elaborated further on herein, the present invention contemplates therapeutic intervention to regenerate or repair cartilage upon the damage of cartilage or absence of properly formed cartilage in any of various orthopedic and rheumatologic conditions including degenerative connective tissue diseases or in the event of physical trauma. Conditions or instances in which cartilage is damaged, absent or abnormal are suitable for the methods and therapies of the present invention. Examples of such conditions include, but are not limited to, fracture nonunion, large segmental bone or cartilage defects, ostoarthritis, collagen disorders, dwarfism including camptomelic dysplasia, pseudoachondroplasia, multiuple epiphyseal dysplasia.

The naked DNA constructs of the present invention can be used to create or supplement connective tissue as required. In some cases, this will be to repair existing defects, for example, worn or torn cartilage in joint linings. In other cases, it may be to create new tissue that performs a distinct function, such as to block tubes such as the fallopian tubes or vas derens, or to decrease reflux due to urine leakage arising from incorrect placement of the ureter into the bladder. The selection of the construct and/or composition will in many cases be determined by the function to be achieved. Examples of situations in which new connective tissue is particularly desirable, in addition to cartilage replacement or supplementation, include reconstruction of the spine, pubic symphysis or temporomandibular joint (TMJ).

In some cases, it may be desirable to induce a mixed cell tissue, for example, in breast reconstruction. Breast tissue is naturally composed of fat, cartilage and other connective tissue, muscle and other tissues. New breast tissue can be formed by implanting the naked DNA of the present invention in cells, including for example, mesenchymal cells in a polymeric carrier in a fascial plane formed of muscle cells, fat, fibroblasts, and cartilage.

Furthermore, although the invention is directed predominantly to methods for the production of new cartilage tissue in humans, the invention may also be practiced so as to produce new cartilage tissue in any mammal in need thereof, including horses, dogs, cats, sheep, pigs, among others. The treatment of such animals is intended to fall within the scope of the invention.

As used herein, “pg” means picogram, “ng” means nanogram, “ug” or “μg” mean microgram, “mg” means milligram, “ul” or “μl” mean microliter, “ml” means milliliter, “l” means liter.

Another feature of this invention is the expression of the DNA sequences disclosed herein. As is well known in the art, DNA sequences may be expressed by operatively linking them to an expression control sequence in an appropriate expression vector and employing that expression vector to transform an appropriate unicellular host.

Such operative linking of a DNA sequence of this invention to an expression control sequence, of course, includes, if not already part of the DNA sequence, the provision of an initiation codon, ATG, in the correct reading frame upstream of the DNA sequence.

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, pMB9 and their derivatives, plasmids such as RP4; phage DNAS, e.g., the numerous derivatives of phage λ, e.g., NM989, and other phage DNA, e.g., M13 and filamentous single stranded phage DNA; yeast plasmids such as the 2□ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Any of a wide variety of expression control sequences—sequences that control the expression of a DNA sequence operatively linked to it—may be used to express the DNA sequences of this invention. Such useful expression control sequences include, for example, the early or late promoters of SV40, CMV, vaccinia, polyoma or adenovirus, the lac system, the trp system, the TAC system, the TRC system, the LTR system, the major operator and promoter regions of phage λ, the control regions of fd coat protein, the promoter for 3-phosphoglycerate kinase or other glycolytic enzymes, the promoters of acid phosphatase (e.g., Pho5), the promoters of the yeast-mating factors, and other sequences known to control the expression of genes of prokaryotic or eukaryotic cells or their viruses, and various combinations thereof.

A wide variety of unicellular host cells are also useful in expressing the DNA sequences of this invention. These hosts may include well known eukaryotic and prokaryotic hosts, such as strains of E. coli, Pseudomonas, Bacillus, Streptomyces, fungi such as yeasts, and animal cells, such as CHO, R1.1, B—W and L-M cells, African Green Monkey kidney cells (e.g., COS 1, COS 7, BSC1, BSC40, and BMT10), insect cells (e.g., Sf9), and human cells and plant cells in tissue culture.

It will be understood that not all vectors, expression control sequences and hosts will function equally well to express the DNA sequences of this invention. Neither will all hosts function equally well with the same expression system. However, one skilled in the art will be able to select the proper vectors, expression control sequences, and hosts without undue experimentation to accomplish the desired expression without departing from the scope of this invention

In selecting an expression control sequence, a variety of factors will normally be considered. These include, for example, the relative strength of the system, its controllability, and its compatibility with the particular DNA sequence or gene to be expressed, particularly as regards potential secondary structures. Suitable unicellular hosts will be selected by consideration of, e.g., their compatibility with the chosen vector, their secretion characteristics, their ability to fold proteins correctly, and their fermentation requirements, as well as the toxicity to the host of the product encoded by the DNA sequences to be expressed, and the ease of purification of the expression products.

Considering these and other factors a person skilled in the art will be able to construct a variety of vector/expression control sequence that will permit construction oif the naked DNA of the invention and promote expression of the DNA sequences to generate bioactive agent(s) of this invention on implantation to a cartilage defect site or on transformation of cartilage or chondroyte cells or progenitor cells.

It is further intended that analogs of the bioactive agent may be constructed for expression from the nucleotide sequences and are within the scope of the present invention. Analogs, such as muteins, can be produced by standard site-directed mutagenesis of the bioactive agent(s) coding sequences. Analogs exhibiting “cartilage regeneration and/or repair activity” may be identified by known in vivo and/or in vitro assays.

As mentioned above, a DNA sequence encoding a bioactive agent(s) can be prepared synthetically rather than cloned. The DNA sequence can be designed with the appropriate codons for the bioactive agent(s) amino acid sequence. In general, one will select preferred codons for the intended host if the sequence will be used for expression. The complete sequence is assembled from overlapping oligonucleotides prepared by standard methods and assembled into a complete coding sequence. See, e.g., Edge, Nature, 292:756 (1981); Nambair et al., Science, 223:1299 (1984); Jay et al., J. Biol. Chem., 259:6311 (1984).

Synthetic DNA sequences allow convenient construction of genes which will express bioactive agent(s) analogs or “muteins”. Alternatively, DNA encoding muteins can be made by site-directed mutagenesis of native bioactive agent(s) genes or cDNAs, and muteins can be made directly using conventional polypeptide synthesis.

A general method for site-specific incorporation of unnatural amino acids into proteins is described in Christopher J. Noren, Spencer J. Anthony-Cahill, Michael C. Griffith, Peter G. Schultz, Science, 244:182-188 (April 1989). This method may be used to create analogs with unnatural amino acids.

The naked DNA may contain or the bioactive agent may be expressed to include a label, unrelated peptide or indicator, for instance an unrelated peptide, an antigenic site, a compound or peptide conferring antibiotic resistance or sensitivity, an enzyme, and/or a radioactive element which can be utilized as an indicator of the presence of the naked DNA and/or the expression of the biactive agent(s) in a cellular sample or in vivo. The presence or amount of the label, unrelated peptide or indicator may be examined by known techniques, which may vary with the nature of the label attached. In the instance where a radioactive label, such as the isotopes ³H, ¹⁴C, ³²P, ³⁵S, ³⁶Cl, ⁵¹Cr, ⁵⁷Co, ⁵⁸ Co, ⁵⁹Fe, ⁹⁰Y, ¹²⁵I, ¹³¹I, and ¹⁸⁶Re are used, known currently available counting procedures may be utilized. In the instance where the label is an enzyme, detection may be accomplished by any of the presently utilized calorimetric, spectrophotometric, fluorospectrophotometric, amperometric or gasometric techniques known in the art.

A number of fluorescent materials are known and can be utilized as labels. These include, for example, fluorescein, rhodamine, auramine, Texas Red, AMCA blue and Lucifer Yellow. A particular detecting material is anti-rabbit antibody prepared in goats and conjugated with fluorescein through an isothiocyanate.

Enzyme labels are likewise useful, and can be detected by any of the presently utilized calorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Many enzymes which can be used in these procedures are known and can be utilized. The preferred are peroxidase, β-glucuronidase, β-D-glucosidase, β-D-galactosidase, urease, glucose oxidase plus peroxidase and alkaline phosphatase. U.S. Pat. Nos. 3,654,090; 3,850,752; and 4,016,043 are referred to by way of example for their disclosure of alternate labeling material and methods.

Alternatively, or in addition, the naked DNA may contain or the bioactive agent may be expressed to include a control element or transcription element or binding site, etc., whereby the expression of the encoded agent(s) or other unrelated peptide can be modulated or otherwise controlled, i.e. activated or inactivated. Such a construct provides an inducible or extinguishable system, whereby the enhancement of or extent of cartilage regeneration or repair can be modulated, turned on or turned off in certain circumstances. Such a system may be constructed to respond to the addition of a factor or chemical agent, for instance, which can be administered or added.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### EXAMPLE 1

Normal joint function requires a smooth articular surface composed of hyaline cartilage. When damaged, hyaline cartilage has limited ability to repair itself, and so what may seem like a small lesion, if left untreated, can hinder one's ability to move free from pain and cause joint surface deterioration leading to arthritis.

While conventional therapeutic options may temporarily alleviate clinical symptoms, none yet succeeds in regenerating cartilage that exhibits the structural, biochemical, and biomechanical properties of healthy articular tissue. Several therapeutic agents have been identified that have the potential to stimulate cartilage repair, among them growth factors and transplanted cells, but the great expense and lack of adequate delivery mechanisms associated with their use prevent their widespread clinical application.

Another approach is to employ gene therapy, which often uses viral vectors to carrying desired genes into host cells. This type of gene transfer is usually transient, however, and repeated attempts are short-circuited when the immune system recognizes and destroys the invading virus. Vector-induced inflammation of host tissues is also common, and there is also a risk that the cells' own genome may be compromised.

The present studies take a new approach to gene therapy for treating damaged cartilage: a gene (in one example, bone morphogenetic protein-2) is implanted in collagen sponges and then transferred as a naked plasmid DNA construct (a double-stranded loop of nucleotide bases, also known as a plasmid) to the injury site. The early repair cells that populate a full-thickness articular cartilage defect incorporate the implanted naked plasmid DNA and, once transfected, serve as local bioreactors, transiently producing peptide factors that stimulate the cartilage to regenerate virtually normal tissue. This approach (1) eliminates the need for a surgical procedure to harvest cartilage cells (a feature of most other techniques) and thus elminates the potential for attendant morbidity at the harvest site; and (2) circumvents the risks of inflammation and immunogenic response associated with the use of viral vectors. In short, the use of naked plasmid DNA as a vehicle for transferring therapeutic genes is a potentially important clinical means of facilitating articular cartilage regeneration that is both safe and cost-effective.

This study seeks to develop a new, cost-effective regional gene therapy technique using naked plasmid DNA to facilitate cartilage repair and prevent its degeneration to arthritis. Nonviral vectors are far more amenable to tissue engineering techniques for circumventing immunogenic and other toxic effects and for overcoming the production challenges associated with viral vectors. These artificial gene vectors can be directly implanted into the body to carry therapeutic genes directly to the target cells (hence “regional” therapy). The simplicity of this direct, nonviral approach should help facilitate clinical trials and subsequent FDA approval. This technology is considered safe because it does not result in the infection and cell damage observed with the use of viral vectors or other gene transfer technologies.

The underlying tissue engineering and gene therapy techniques of this system, which seeks to generate biological repair of cartilage, constitute a revolutionary approach to the problem of treating injury and degeneration of this tissue. Previous systems that employ growth factors to treat injured cartilage and prevent subsequent arthritic degeneration have been hampered by the absence of a suitable delivery system. The use of regional naked DNA plasmids encoding stimulatory factors for cartilage repair represents a local gene delivery system that would solve this problem, and would also be simple, relatively inexpensive, and have widespread musculoskeletal applications.

Several peptide growth and differentiation factors have been identified that appear to control cellular events associated with cartilage formation and repair. The BMPs comprise a family of soluble extracellular proteins that can control osteogenic cell fate, initiate chondrogenesis, and maintain the chondrocyte phenotype. Fetal osteoblasts in vitro and in vivo normally express BMP genes [30], and recombinant BMP proteins initiate cartilage and bone progenitor cell differentiation [31, 32]. BMP-2 is a potent stimulator of articular cartilage proteoglycan synthesis in vitro and in vivo [33, 34]. BMP-3 and BMP-4 prevent chondrocyte dedifferentiation and maintain the differentiated phenotype in vitro [35].

Growth and differentiation factors have been used to facilitate articular cartilage repair [36-38]. Relatively large doses (microgram amounts) are required to stimulate new cartilage formation in animals, however, raising the concern that future human therapies will be expensive and may possess an increased risk of toxicity. As an alternative, regional gene therapy will be more cost-effective, because production of DNA is much less expensive than traditional methods of protein production [39]. Gene therapy shows promise as a more efficient way to deliver chondrogenic factors in vivo since the cell-mediated synthesis and delivery are likely associated with efficient targeting to cell surface receptors; consequently, less protein will be required to achieve a clinically desirable effect [40-42]. Local delivery of cDNAs encoding several potentially antiarthritic genes has been achieved by ex vivo transfer with an adenovirus or retrovirus [43-48].

Gene therapy for arthritis has focused on the use of viral vectors to introduce the desired gene, typically one that encodes an inhibitor of cartilage degeneration (e.g., members of the interleukin family) into synoviocytes or chondrocytes ex vivo. The transfected cells are then implanted with the expectation that they will express the desired gene and prevent further deterioration [49]. Concerns have been voiced regarding the use of retroviruses, however, including the risk of insertional mutagenesis, the difficulty in targeting specific tissues, and immunogenicity to viral particles. The studies described herein take a radically different approach. Rather than harvest donor cells, transfect them ex vivo, and implant them, we implant, at an injury site, naked plasmid DNA vectors expressing a cartilage morphogen, in one instance bone morphogenetic protein-2 (BMP-2). The naked plasmid DNA constructs can be delivered to the site in a scaffold matrix. This eliminates the need for a surgical procedure to harvest cells, as well as the attendant risk of morbidity, and will eliminate the significant concerns raised by the use of viral vectors. Furthermore, use of a gene for a factor that shows early evidence of enhancing cartilage repair [36, 50] increases the likelihood that a hyaline-like surface will be restored.

We describe a new regional gene therapy technique using naked plasmid DNA. Stimulating cartilage growth by direct naked plasmid DNA transfer from a matrix has several advantages: (i) Producing and purifying DNA constructs is easier than producing and purifying protein; (ii) Matrices can act as structural scaffolds that themselves promote wound healing by directing cell migration to the site of drug delivery; (iii) Matrices can protect and target plasmid DNA and allow for its sustained release [51, 52]; (iv) Gene transfer may be an advantageous method of drug delivery for molecules that normally undergo complex biosynthetic processing [53]; (v) Local production and cell-mediated growth factor delivery to signaling receptors is likely to be more efficient than exogenous delivery of recombinant protein; and (vi) a naked plasmid DNA gene transfer system can deliver even more than one gene encoding different cartilage-promoting factors with synergistic effects that represent considerable therapeutic potential and significance.

Naked gene transfer into the tissues of living organisms represents a promising gene delivery system because it has both fundamental and therapeutic implications. While a potential disadvantage of naked plasmid DNA gene therapy is that it has a lower transfection rate than viral vectors, it appears from our preliminary studies that for this application the proposed technique will be sufficient for transferring therapeutic genes. It has been reported that naked plasmid DNA can be effectively transferred and expressed in vivo by direct injection into mouse brain [54], melanoma [55], and skin [56]. The potential therapeutic usefulness of naked plasmid DNA gene therapy is the subject of several studies. Fang et al. [57] showed that both BMP-4 expression vector alone and a mixture of naked DNA plasmids expressing BMP-4 and parathyroid-hormone-related protein can stimulate new bone formation in a rat osteotomy model. The potential therapeutic role of TGF-β was investigated in a rat model for human rheumatoid arthritis [58]. In this study a single dose of naked plasmid DNA encoding human TGF-β was injected intramuscularly in rats with induced arthritis. Rats treated with the plasmid DNA encoding TGF-β, but not vector DNA, administered at the peak of the acute phase profoundly suppressed the subsequent evolution of chronic erosive joint disease. Moreover, delivery of the TGF-β DNA even as the chronic phase commenced virtually eliminated subsequent inflammation and arthritis. Increases in TGF-β protein were detected in the circulation of TGF-β-DNA-treated animals, consistent with the observed therapeutic effects being TGF-β-dependent. Jones and co-workers [59] induced ulcers in rats and then injected tissues surrounding the ulcer site with naked plasmid DNA of angiogenic factors. A single local injection of naked plasmid DNA encoding vasoendothelial growth factor and angiopoietin-1 significantly increased neovascularization and accelerated ulcer healing.

### RESULTS AND DISCUSSION

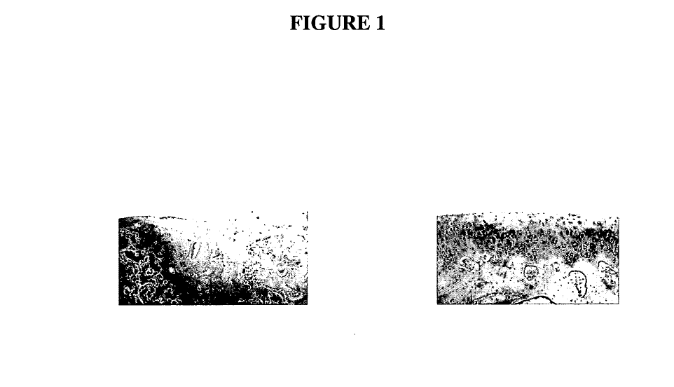

A study was conducted using recombinant human BMP-2 to enhance cartilage repair [16]. Full-thickness articular cartilage defects in the rabbit knees were grafted with either 6 μg of BMP-2 in a collagen sponge or collagen sponge alone (control). At 6 weeks, animals were sacrificed and histological sections were scored with a modification of O'Driscoll's grading system (0-27; 27 best) for cartilage repair [4, 17]. Mean repair scores for the BMP-2-Treated or control group were 21.9±2.2 and 12.3±5.5 respectively (p>0.5). Repair tissue in the control group was disorganized, hypercellular, and fibrillated (FIG. 1, left). Sponges containing BMP-2 induced a high-quality hyaline-appearing repair (FIG. 1, right). In nearly all cases, the neocartilage did not overgrow the surface of the native tissue or display a greater thickness (as is often observed in fibrocartilaginous repair). Surfaces were smooth, with no evidence of fibrillation, and columnar organization of chondrocytes was apparent. Safranin O staining indicated a glycosaminoglycan distribution similar to that of the normal adjacent tissue.

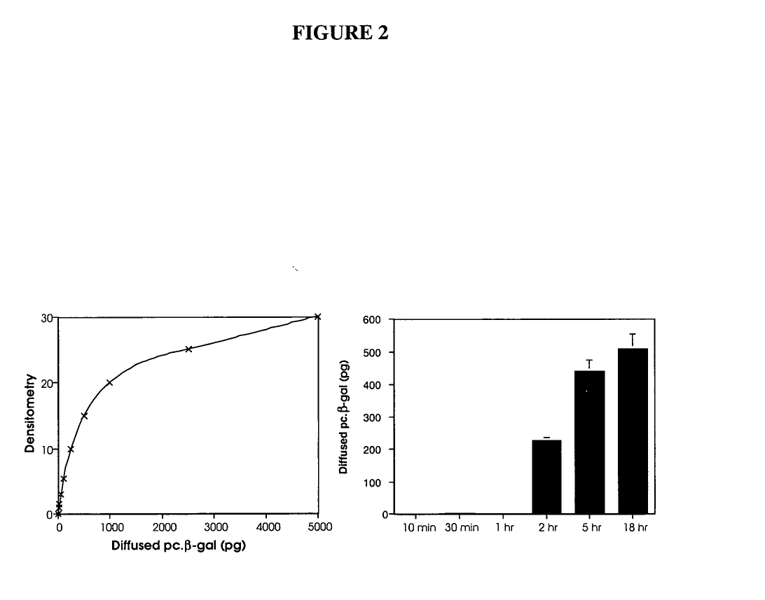

In preparation for in vivo studies with naked plasmid DNA, the diffusion kinetics of the plasmid DNA applied to collagen sponges were examined using an in vitro assay. Five micrograms of a plasmid for beta-galactosidase (pc.βgal) was applied to 3-mm-diameter collagen sponges. The DNA-containing sponges were fit into a 3-mm hole in a polystyrene slab to mimic an in vivo full-thickness cartilage defect. Then the polystyrene slab-sponge construct and incubated at 37° C. with constant agitation with human synovial fluid. Synovial fluid was sampled at 10 min, 30 min, and 1, 2, 5, and 18 hr. Each sample was then used as template in polymerase chain reaction (PCR) amplification with a primer set specific for β-gal cDNA. PCR products were then examined in a 1% agarose gel, photographed, and densitometry-quantified. The data showed a continuous slow diffusion rate of plasmid DNA from the sponge for up to 18 hr, at which time the sponge still retained more than 90% of the plasmid DNA (FIG. 2).

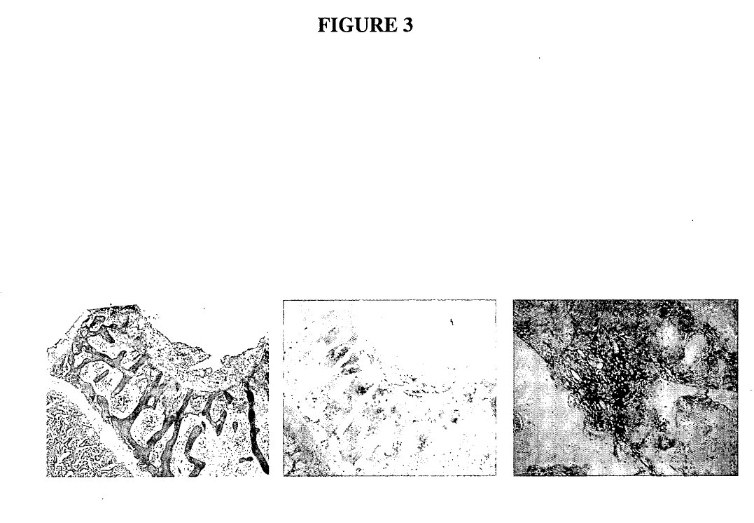

To test in vivo whether mesenchymal cells at the defect site can take up and express the plasmid DNA, a marker gene (pc.βgal) was used. A type I collagen sponge containing approximately 70 μg of lipopolysaccharide (LPS)-free pc.βgal DNA was surgically pressed into defects in skeletally mature (9-month-old) male New Zealand White rabbits. One week postsurgery, rabbits were sacrificed. Sections through the defect area were immunostained with murine monoclonal antibody to β-gal (β-gal). Results (FIG. 3) demonstrated numerous mesenchymal cells deep to the defect were expressing β-galactosidase. Differentiated bone cells (both osteoblasts and osteocytes), chondrocytes, and synoviocytes were negative to staining.

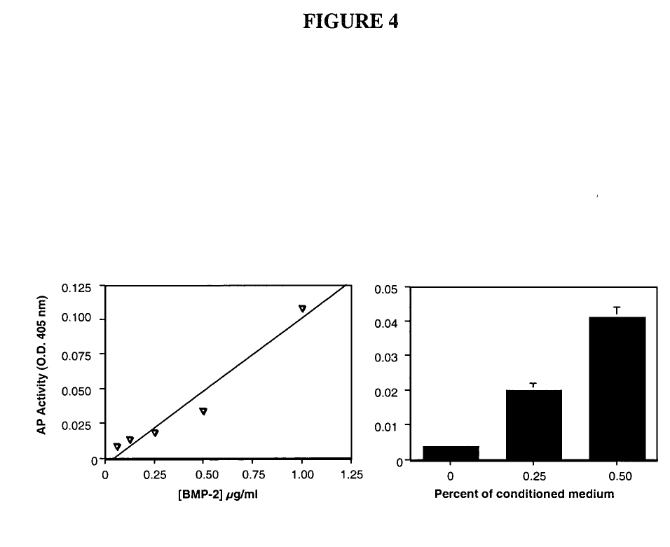

A BMP-2 cDNA (Genetic Institute) was cloned into pc.DNA3 (+) (Invitrogen) and the resultant plasmid named pc.hBMP-2. The BMP-2 cDNA sequence is depicted in FIG. 6 (SEQ ID NO: 1) and encodes a BMP-2 polypeptide depicted in FIG. 7 (SEQ ID NO: 2). The BMP-2 sequences correspond to the Genbank entry NM_{—}001200 as provided at the National Center for Biotechnology Information website (ncbi.nlm.nih.gov). The plasmid, pc.DNA3 (+), is designed for high-level stable and transient expression in a wide range of mammalian hosts; it contains human cytomegalovirus immediate-early promoter for high-level expression. pc.hBMP-2 was then transfected into 293T cells using Lipofectamine (Life Technology). At 48 hr posttransfection, the conditioned medium containing the expressed protein was collected. The functional expression of BMP-2 was confirmed using a murine bone marrow stromal cell line (W-20-17) [18, 19]. Briefly, cells were stimulated for 48 hr with either purified BMP-2 protein at varying concentrations (control curve) or pc.hBMP-2 conditioned medium (FIG. 4). Alkaline phosphatase activity was measured at 405 nm on an enzyme-linked immunosorbent assay reader [20] (Sigma) confirmed transfected cell expression of active BMP-2.

### EXAMPLE 2

### Characterization of Naked Plasmid DNA Transfer to Full-Thickness Cartilaginous Defects

In our preliminary in vivo study with marker gene plasmid DNA in a rabbit full-thickness defect model, we detected marker protein in many mesenchymal cells under the defect area 1 wk postsurgery. It is clear that these cells take up the naked DNA plasmid and express protein. This study characterizes the duration of active expression of the naked plasmid DNA. 70 μg of pc.βgal/collagen or collagen sponge with control plasmid are implanted in full-thickness articular cartilage defect in 12 adult (9-month-old) male New Zealand White rabbits and samples analyzed after 3, 7, 10, and 21 days postimplantation (3 rabbits=6 knees per time point). These time points are based on other studies using naked plasmid DNA for regional gene therapy that showed active plasmid for up to 3 weeks [54-59]. At each time point, triplicate samples are immunostained with murine monoclonal antibody against β-gal and undergo in situ hybridization with a riboprobe specific for β-gal to determine which cells specifically contain the naked plasmid DNA gene and express the marker.

Preparation of LPS-free plasmid DNAs: Plasmid DNA is synthesized in bacteria and traditional DNA isolation protocols are contaminated by toxic doses of endotoxin (LPS) [60]. An efficient protocol of Wicks et al. [60] is utilized to prepare plasmid DNA with 10,000-fold reduction in LPS contamination for in vivo studies [61].

In vivo examination of marker gene expression: Immunohistochemistry and In situ hybridization: The procedure described below has been established in our laboratories [63]. All samples are fixed for in fresh 4% paraformaldehyde (PF) in phosphate-buffered saline (PBS) then decalcified at 4° C., embedded in paraffin, and serially sectioned (6 μm thick). Sections are deparaffinized and rehydrated. For immunostaining, sections are digested for 1 hr at 37° C. with 0.1-U/ml chondroitinase ABC (Sigma) in order to increase antibody permeability. Sections are probed with murine monoclonal antibody to β-gal or control murine monoclonal antibody, followed by incubation with biotinylated goat antimouse immunoglobulin antibody (Biogenex). Following washing, sections are incubated with alkaline phosphatase conjugated with streptavidin (Biogenex) and then developed with Vector Red I (Vector Lab) and counterstained with Mayer's Hematoxylin (Sigma).

For in situ hybridization, sections are hydrated in buffer containing 0.5% casein to block nonspecific binding. Single-stranded antisense and sense digoxigenin-labeled riboprobes are synthesized for β-gal. A ClaI-EcoRV fragment (290 bp) of pc.β-gal is cloned into the same sites in a pBSK plasmid (Invitrogen) and the resulting vector called pB(CE) β-gal. Next pB(CE) β-gal is used as template for transcription of both sense and antisense digoxinine-labeled β-gal riboprobes. Specimens are rehydrated with serial washes of 100%, 95%, and 70% ethanol followed by DEPC-treated distilled water. Prehybridization is performed with serial washes of DEPC-treated PBS (pH 7.4) (D-PBS), D-PBS plus 0.3% Triton X-100, and D-PBS alone; sections are permeabilized with 100 mM Tris-HCl, 50 mM EDTA, pH 8.0 (TE Buffer) containing RNase-free proteinase-K (1 μg/ml). Postfixation is performed with D-PBS containing 4% paraformaldehyde followed by D-PBS washes. Each section is washed in prehybridization buffer containing 50% (v/v) deionized formamide. Each section is probed with hybridization buffer containing 10 ng of either sense or antisense digoxigenin-labeled β-gal riboprobe at 42° C. overnight in a humid chamber. For posthybridization, slides are washed and unbound single-stranded probe-digested in buffer containing 20 μg/ml RNase A. Sections are washed and blocked with the last buffer containing 2% sheep serum. Bound probe is detected by an alkaline-phosphatase-linked sheep antidigoxigenin antibody and the addition of nitroblue tetrazolium and 5-bromo-4-chloro-3-indolyphosphate as a substrate (Boehringer-Mannheim). Cells containing the plasmid to β-gal exhibit a dark blue-black reaction product by light microscopy.

### EXAMPLE 3

### Regional Gene Therapy Using Naked Plasmid DNA Encoding the BMP-2 Gene to Promote Hyaline-Like Articular Cartilage Repair

To document with statistical significance (p<0.05; power=80%) the efficacy of this technique, 9 knees per group are performed. In this study, we examine the cartilage repair at 3 months (18 animals=36 knees) postsurgery in four treatment groups: sponge only, sponge with 6 μg of recombinant human BMP-2, sponge with control plasmid pc.DNA3 (+), and sponge with naked plasmid DNA encoding BMP-2. Repair samples are examined by gross examination, histology (H&E staining), Safranin O staining for proteoglycans, immunostaining (with antibodies to type II collagen and cartilage oligomeric matrix protein).

Evaluation of cartilage repair in the defects: Cartilage defects are examined macroscopically at sacrifice. Similarity of appearance of the repair cartilage to that of normal adjacent cartilage is assessed based on color, texture, and shape of cartilage surface. For histological evaluation, sections from the center of each defect are stained with either H&E or Safranin O and evaluated using a modified version of a standard histological grading system [4, 17] for cartilage repair.

Immunohistochemistry: Samples are immunostained using biotinylated polyclonal antiserum to type II collagen (Rockland, Gilbertsville, Pa.), COMP, or preimmune rabbit serum (control) as described above.

### EXAMPLE 4

An eight (8) week rabbit defect study was conducted in which repair was assessed in vivo in full thickness cartilaginous defects in rabbits. Rabbits were treated with Naked-BMP-2, plasmid control or BMP-2 protein, with four (4) knees per group (wherein each of the two knees of a single rabbit were treated with different material).

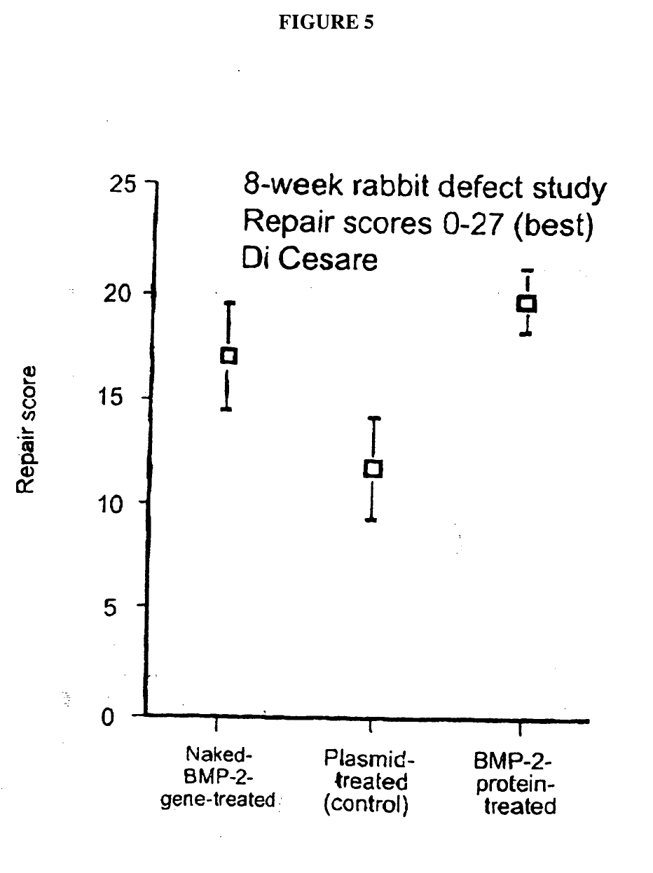

Plasmid was administered in the amount of 70 ug. Animals were scored for repair at eight (8) weeks post implantation as described above. The results are depicted in FIG. 5. Each of the Naked-BMP-2 and BMP-2 protein treated groups showed significant repair over the plasmid control, but were not significantly different from each other.

### EXAMPLE 5

### Regional Gene Therapy Using Naked Plasmid DNA Encoding The BMP-2 Gene To Promote Hyaline-Like Articular Cartilage Repair

Methods:

Studies were conducted according to a protocol approved by the IACUC. To document the efficacy of this technique, 30 knees (15 animals, bilateral defects) were implanted, and animals sacrificed 3 months postoperatively. A medial parapatellar incision was made, and the patella dislocated laterally. A 3-mm-diameter full-thickness midtrochlear defect was drilled at low speed under continuous irrigation. The implant, consisting of a 3.5 mm-diameter bovine type I collagen sponge (Helistat, Integra LifeSciences, Palo Alto, Calif.) with one of four treatments, was press-fit into the defect. The four treatment groups used were: collagen implant alone, collagen with 6 μg of recombinant human BMP-2 (R & D Systems, Minneapolis, Minn.), collagen with 70 μg control plasmid DNA encoding beta-galactosidase, and collagen with 70 μg naked plasmid DNA encoding BMP-2. The patella was reduced, and the wound closed in layers. Animals were allowed free cage movement and food and water ad libitum.

Evaluation of cartilage repair in the defects. For histological evaluation, sections were stained with H&E or Safranin O and evaluated using a modified O'Driscoll histological grading system for cartilage repair [40, 45]. Criteria included percentage of hyaline cartilage, surface regularity, thickness, bonding to host cartilage and bone, freedom from degenerative changes in host cartilage, reconstitution of subchondral bone, and Safranin O staining.

Statistical Analysis. Analysis of variance (ANOVA) was used to analyze the histological and mechanical testing data. The Tukey test was used for all post hoc multiple comparisons.

Results:

The results of this study are presented in TABLE 1.

TABLE 1

| | | | | β-gal |
|---|---|---|---|---|
| | BMP protein | BMP plasmid | Collagen alone | plasmid |
| Mean | 20.25 | 18.81818182 | 15.833333333 | 14.77272727 |
| Standard deviation | 2.4634 | 3.7899 | 5.2559 | 4.7821 |
| (SD) | | | | |
| Sample size (N) | 12 | 11 | 9 | 11 |
| Std. error of mean | 0.71111 | 1.1427 | 1.752 | 1.4418 |
| (SEM) | | | | |
| Lower 95% conf. | 18.685 | 16.272 | 11.793 | 11.56 |
| limit | | | | |
| Upper 95% conf. | 21.815 | 21.364 | 19.873 | 17.985 |
| limit | | | | |
| Minimum | 16.000 | 12.000 | 7.000 | 7.000 |
| Median (50th | 21.000 | 20.000 | 18.000 | 15.500 |
| Percentile) | | | | |
| Maximum | 23.000 | 24.000 | 22.000 | 21.000 |
| Normality test KS | 0.2863 | 0.1679 | 0.3266 | 0.1553 |
| Normality test P | >0.10 | >0.10 | >0.10 | >0.10 |
| value | | | | |
| Passed normality | Yes | Yes | Yes | Yes |
| test? | | | | |

Significant Difference (<.05)
BMP protein + β-gal plasmid, collagen alone
BMP plasmid + β-gal plasmid (but not collagen)

### REFERENCES

1. Buckwalter J A, Lohmander S: Operative treatment of osteoarthrosis. Current practice and future development. J Bone Joint Surg Am, 1994. 76(9):1405-18.

2. Buckwalter J A, Mow V C, Ratcliffe A: Restoration of Injured or Degenerated Articular Cartilage. J Am Acad Orthop Surg, 1994. 2(4):192-201.

3. Homminga G N, Bulstra S K, Bouwmeester P S, van der Linden A J: Perichondral grafting for cartilage lesions of the knee. J Bone Joint Surg Br, 1990. 72(6):1003-7.

4. O'Driscoll S W, Keeley F W, Salter R B: The chondrogenic potential of free autogenous periosteal grafts for biological resurfacing of major full-thickness defects in joint surfaces under the influence of continuous passive motion. An experimental investigation in the rabbit. J Bone Joint Surg Am, 1986. 68(7):1017-35.

5. Brittberg M, Lindahl A, Nilsson A, Ohlsson C, Isaksson O, Peterson L: Treatment of deep cartilage defects in the knee with autologous chondrocyte transplantation. N Engl J Med, 1994. 331(14):889-95.

6. Caplan A I, Elyaderani M, Mochizuki Y, Wakitani S, Goldberg V M: Principles of cartilage repair and regeneration. Clin Orthop, 1997(342):254-69.

7. Joyce M E, Roberts A B, Sporn M B, Bolander M E: Transforming growth factor-beta and the initiation of chondrogenesis and osteogenesis in the rat femur. J Cell Biol, 1990. 110(6):2195-207.

8. Hunziker E B: Growth-factor-induced healing of partial-thickness defects in adult articular cartilage. Osteoarthritis Cartilage, 2001. 9(1):22-32.

9. Hunziker E B, Rosenberg L C: Repair of partial-thickness defects in articular cartilage: cell recruitment from the synovial membrane. J Bone Joint Surg Am, 1996. 78(5):721-33.

10. Kato Y, Gospodarowicz D: Sulfated proteoglycan synthesis by confluent cultures of rabbit costal chondrocytes grown in the presence of fibroblast growth factor. J Cell Biol, 1985. 100(2):477-85.

11. Osborn K D, Trippel S B, Mankin H J: Growth factor stimulation of adult articular cartilage. J Orthop Res, 1989. 7(1):35-42.

12. Wozney J M: The bone morphogenetic protein family and osteogenesis. Mol Reprod Dev, 1992. 32(2): 160-7.

13. Reddi A H: Cartilage morphogenesis: role of bone and cartilage morphogenetic proteins, homeobox genes and extracellular matrix. Matrix Biol, 1995. 14(8):599-606.

14. Centrella M, Horowitz M C, Wozney J M, McCarthy T L: Transforming growth factor-beta gene family members and bone. Endocr Rev, 1994. 15(1):27-39.

15. Sumner D R, Turner T M, Purchio A F, Gombotz W R, Urban R M, Galante J O: Enhancement of bone ingrowth by transforming growth factor-beta. J Bone Joint Surg Am, 1995. 77(8):1135-47.

16. Frenkel S R, Saadeh P B, Mehrara B J, Chin G S, Steinbrech D S, Brent B, Gittes G K, Longaker M T: Transforming growth factor beta superfamily members: role in cartilage modeling. Plast Reconstr Surg, 2000. 105(3):980-90.

17. O'Driscoll S W, Marx R G, Beaton D E, Miura Y, Gallay S H, Fitzsimmons J S: Validation of a simple histological-histochemical cartilage scoring system. Tissue Eng, 2001. 7(3):313-20.

18. Bahamonde M E, Lyons K M: BMP3: to be or not to be a BMP. J Bone Joint Surg Am, 2001. 83-A Suppl 1(Pt 1):S56-62.

19. Thies R S, Bauduy M, Ashton B A, Kurtzberg L, Wozney J M, Rosen V: Recombinant human bone morphogenetic protein-2 induces osteoblastic differentiation in W-20-17 stromal cells. Endocrinology, 1992. 130(3):1318-24.

20. Galanti B, Nardiello S, Russo M, Giusti G: A comparative study of the Bessey method and Hausamen method for serum alkaline phosphatase determination. Enzyme, 1976. 21(4):374-8.

21. Herndon J H, Davidson S M, Apazidis A: Recent socioeconomic trends in orthopaedic practice. J Bone Joint Surg Am, 2001. 83-A(7):1097-105.

22. Praemer A, Furner S, Rice D: Musculoskeletal Conditions in the U.S. 1999, Rosemont, Ill.: American Academy of Orthopaedic Surgeons.

23. Mankin H, Mow V, Buckwalter J, Ianotti J, Ratcliff A, Form and function of articular cartilage.: in Orthopaedic Basic Science, Simon S, Editor. 1994, American Academcy of Orthopaedic Surgeons: Chicago. p. 2-44.

24. Mankin H J: The response of articular cartilage to mechanical injury. J Bone Joint Surg Am, 1982. 64(3):460-6.

25. Trippel S B: Autologous chondrocyte transplantation. N Engl J Med, 1995. 332(8):539-40.

26. Minas T, Spector M, Shortroff S, Hsu H, Chi T: New animal, human data reported for autologous chondrocyte transplants. Orthop Today, 1996. 16:18-19.

27. Peterson L, Lindahl A: Chondrocyte transplantation technique described. Orthop Today, 1996. 16:1-9.

28. Ellis E, 3rd, Carlson D S: Histologic comparison of the costochondral, sternoclavicular, and temporomandibular joints during growth in Macaca mulatta. J Oral Maxillofac Surg, 1986. 44(4):312-21.

29. Wakitani S, Imoto K, Yamamoto T, Saito M, Murata N, Yoneda M: Human autologous culture expanded bone marrow mesenchymal cell transplantation for repair of cartilage defects in osteoarthritic knees. Osteoarthritis Cartilage, 2002. 10(3):199-206.

30. Katagiri T, Yamaguchi A, Komaki M, Abe E, Takahashi N, Ikeda T, Rosen V, Wozney J M, Fujisawa-Sehara A, Suda T: Bone morphogenetic protein-2 converts the differentiation pathway of C2C12 myoblasts into the osteoblast lineage. J Cell Biol, 1994. 127(6 Pt 1):1755-66.

31. Reddi A H: Regulation of cartilage and bone differentiation by bone morphogenetic proteins. Curr Opin Cell Biol, 1992. 4(5):850-5.

32. Hill D J, Logan A: Peptide growth factors and their interactions during chondrogenesis. Prog Growth Factor Res, 1992. 4(1):45-68.

33. Kale S, Biermann S, Edwards C, Tarnowski C, Morris M, Long M W: Three-dimensional cellular development is essential for ex vivo formation of human bone. Nat Biotechnol, 2000. 18(9):954-8.

34. Glansbeek H L, van Beuningen H M, Vitters E L, Morris E A, van der Kraan P M, van den Berg W B: Bone morphogenetic protein 2 stimulates articular cartilage proteoglycan synthesis in vivo but does not counteract interleukin-1alpha effects on proteoglycan synthesis and content. Arthritis Rheum, 1997. 40(6):1020-8.

35. Luyten F P, Yu Y M, Yanagishita M, Vukicevic S, Hammonds R G, Reddi A H: Natural bovine osteogenin and recombinant human bone morphogenetic protein-2B are equipotent in the maintenance of proteoglycans in bovine articular cartilage explant cultures. J Biol Chem, 1992. 267(6):3691-5.

36. Sellers R S, Peluso D, Morris E A: The effect of recombinant human bone morphogenetic protein-2 (rhBMP-2) on the healing of full-thickness defects of articular cartilage. J Bone Joint Surg Am, 1997. 79(10):1452-63.

37. Ripamonti U, Duneas N, Van Den Heever B, Bosch C, Crooks J: Recombinant transforming growth factor-beta1 induces endochondral bone in the baboon and synergizes with recombinant osteogenic protein-1 (bone morphogenetic protein-7) to initiate rapid bone formation. J Bone Miner Res, 1997. 12(10):1584-95.

38. Glansbeek H L, van Beuningen H M, Vitters E L, van der Kraan P M, van den Berg W B: Stimulation of articular cartilage repair in established arthritis by local administration of transforming growth factor-beta into murine knee joints. Lab Invest, 1998. 78(2):133-42.

39. Horn N A, Meek J A, Budahazi G, Marquet M: Cancer gene therapy using plasmid DNA: purification of DNA for human clinical trials. Hum Gene Ther, 1995. 6(5):565-73.

40. Musgrave D S, Fu F H, Huard J: Gene therapy and tissue engineering in orthopaedic surgery. J Am Acad Orthop Surg, 2002. 10(1):6-15.

41. Chen Y: Orthopedic applications of gene therapy. J Orthop Sci, 2001. 6(2):199-207.

42. Evans C H, Ghivizzani S C, Smith P, Shuler F D, Mi Z, Robbins P D: Using gene therapy to protect and restore cartilage. Clin Orthop, 2000(379 Suppl):S214-9.

43. Bandara G, Robbins P D, Georgescu H I, Mueller G M, Glorioso J C, Evans C H: Gene transfer to synoviocytes: prospects for gene treatment of arthritis. DNA Cell Biol, 1992. 11(3):227-31.

44. Hung G L, Galea-Lauri J, Mueller G M, Georgescu H I, Larkin L A, Suchanek M K, Tindal M H, Robbins P D, Evans C H: Suppression of intra-articular responses to interleukin-1 by transfer of the interleukin-1 receptor antagonist gene to synovium. Gene Ther, 1994. 1(1):64-9.

45. Otani K, Nita I, Macaulay W, Georgescu H I, Robbins P D, Evans C H: Suppression of antigen-induced arthritis in rabbits by ex vivo gene therapy. J Immunol, 1996. 156(9):3558-62.

46. Roessler B J, Hartman J W, Vallance D K, Latta J M, Janich S L, Davidson B L: Inhibition of interleukin-1-induced effects in synoviocytes transduced with the human IL-1 receptor antagonist cDNA using an adenoviral vector. Hum Gene Ther, 1995. 6(3):307-16.

47. Makarov S S, Olsen J C, Johnston W N, Anderle S K, Brown R R, Baldwin A S, Jr., Haskill J S, Schwab J H: Suppression of experimental arthritis by gene transfer of interleukin 1 receptor antagonist cDNA. Proc Natl Acad Sci U S A, 1996. 93(1):402-6.

48. Evans C H, Robbins P D, Ghivizzani S C, Herndon J H, Kang R, Bahnson A B, Barranger J A, Elders E M, Gay S, Tomaino M M, Wasko M C, Watkins S C, Whiteside T L, Glorioso J C, Lotze M T, Wright T M: Clinical trial to assess the safety, feasibility, and efficacy of transferring a potentially anti-arthritic cytokine gene to human joints with rheumatoid arthritis. Hum Gene Ther, 1996. 7(10):1261-80.

49. Lechman E R, Jaffurs D, Ghivizzani S C, Gambotto A, Kovesdi I, Mi Z, Evans C H, Robbins P D: Direct adenoviral gene transfer of viral IL-10 to rabbit knees with experimental arthritis ameliorates disease in both injected and contralateral control knees. J Immunol, 1999. 163(4):2202-8.

50. Frenkel S R, Toolan B, Menche D, Pitman M I, Pachence J M: Chondrocyte transplantation using a collagen bilayer matrix for cartilage repair. J Bone Joint Surg Br, 1997. 79(5):831-6.

51. Labhasetwar V, Bonadio J, Goldstein S, Chen W, Levy R J: A DNA controlled-release coating for gene transfer: transfection in skeletal and cardiac muscle. J Pharm Sci, 1998. 87(11):1347-50.

52. Labhasetwar V, Levy R J: Implants for site-specific drug delivery. J Appl Biomater, 1991. 2(3):211-2.

53. Yin W, Smiley E, Germiller J, Mecham R P, Florer J B, Wenstrup R J, Bonadio J: Isolation of a novel latent transforming growth factor-beta binding protein gene (LTBP-3). J Biol Chem, 1995. 270(17):10147-60.

54. Schwartz B, Benoist C, Abdallah B, Rangara R, Hassan A, Scherman D, Demeneix B A: Gene transfer by naked DNA into adult mouse brain. Gene Ther, 1996. 3(5):405-11.

55. Liu F, Huang L: Development of non-viral vectors for systemic gene delivery. J Control Release, 2002. 78(1-3):259-66.

56. Hengge U R, Walker P S, Vogel J C: Expression of naked DNA in human, pig, and mouse skin. J Clin Invest, 1996. 97(12):2911-6.

57. Fang J, Zhu Y Y, Smiley E, Bonadio J, Rouleau J P, Goldstein S A, McCauley L K, Davidson B L, Roessler B J: Stimulation of new bone formation by direct transfer of osteogenic plasmid genes. Proc Natl Acad Sci U S A, 1996. 93(12):5753-8.

58. Song X Y, Gu M, Jin W W, Klinman D M, Wahl S M: Plasmid DNA encoding transforming growth factor-betal suppresses chronic disease in a streptococcal cell wall-induced arthritis model. J Clin Invest, 1998. 101(12):2615-21.

59. Jones M K, Kawanaka H, Baatar D, Szabo I L, Tsugawa K, Pai R, Koh G Y, Kim I, Sarfeh I J, Tarnawski A S: Gene therapy for gastric ulcers with single local injection of naked DNA encoding VEGF and angiopoietin-1. Gastroenterology, 2001. 121(5):1040-7.

60. Wicks I P, Howell M L, Hancock T, Kohsaka H, Olee T, Carson D A: Bacterial lipopolysaccharide copurifies with plasmid DNA: implications for animal models and human gene therapy. Hum Gene Ther, 1995. 6(3):317-23.

61. Bayston K F, Cohen J: Bacterial endotoxin and current concepts in the diagnosis and treatment of endotoxaemia. J Med Microbiol, 1990. 31(2):73-83.

62. Bonnerot C, Nicolas J-F: Application of LacZ Genefusions to Postimplantation Development. 1993, San Diego, Calif.: Academic Press.

63. Di Cesare P E, Fang C, Leslie M P, Della Valle C J, Gold J M, Tulli H, Perris R, Carlson C S: Localization and expression of cartilage oligomeric matrix protein by human rheumatoid and osteoarthritic synovium and cartilage. J Orthop Res, 1999. 17(3):437-45.

64. Niederauer G G, Slivka M A, Leatherbury N C, Korvick D L, Harroff H H, Ehler W C, Dunn C J, Kieswetter K: Evaluation of multiphase implants for repair of focal osteochondral defects in goats. Biomaterials, 2000. 21(24):2561-74.

This invention may be embodied in other forms or carried out in other ways without departing from the spirit or essential characteristics thereof. The present disclosure is therefore to be considered as in all aspects illustrate and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

1. A composition for treatment or amelioration of an orthopedic or rheumatologic condition comprising naked DNA encoding one or more of a bioactive agent and a pharmaceutically acceptable excipient.

2. A composition for treatment or amelioration of a degenerative connective tissue disease comprising naked DNA encoding one or more of a bioactive agent and a pharmaceutically acceptable excipient.

3. A composition for treatment, amelioration or repair of a cartilage defect comprising naked DNA encoding one or more of a bioactive agent and a pharmaceutically acceptable excipient.

4. The composition of claim 3 for treatment, amelioration or repair of fracture nonunion, large segmental bone or cartilage defects, osteoarthritis, collagen disorders, dwarfism including camptomelic dysplasia, pseudoachondroplasia, or multiple epiphyseal dysplasia.

5. The composition of any of claims 1-3, further combined with or embedded in a polymeric carrier, biodegradable or biomimetic matrix or a scaffold.

6. The composition of claim 5, wherein:
(a) the biodegradable matrix is selected from polyglycolic acid (PGA), polylactic acid (PLA), hyaluronic acid, catgut suture material, gelatin, cellulose, nitrocellulose, collagen, albumin, fibrin, alginate, cotton, or other naturally-occurring biodegradable materials;
(b) the scaffold is selected from nylon (polyamides), dacron (polyesters), polystyrene, polypropylene, polyacrylates, polyvinyl compounds, polycarbonate (PVC), polytetrafluorethylene (PTFE, teflon), thermanox (TPX), polymers of hydroxy acids such as polylactic acid (PLA), polyglycolic acid (PGA), and polylactic acid-glycolic acid (PLGA), polyorthoesters, polyanhydrides, polyphosphazenes, and polyhydroxyalkanoates, and combinations thereof; and
(c) the polymeric carrier is selected from polymeric mesh or sponge and a polymeric hydrogel.

7. The composition of any of claims 1-3 wherein the bioactive agent is selected from the group of cartilage morphogens and factors or peptides which block inhibitory signals preventing the repair or regeneration of cartilage.

8. The composition of any of claims 1-3 wherein the bioactive agent is a factor selected from the group of cellular growth factors, factors that stimulate chondrogenesis, factors that stimulate migration of stromal cells and factors that stimulate matrix deposition.

9. The composition of claim 9 wherein the factor is selected from the group of an FGF, a TGF, and a BMP.

10. The composition of claim 9 wherein the factor is BMP-2.

11. A method for expressing a bioactive agent in chondrocytes in an animal comprising administering naked DNA encoding one or more bioactive agent to a region in said animal where chondrocytes or chondrocyte progenitors are located such that the chondrocytes or chondrocyte progenitors take up the naked DNA and express the bioactive agent(s) encoded thereby.

12. A method for producing cartilage at a cartilage defect site in an animal comprising administering to the defect site in said animal naked DNA encoding one or more bioactive agent capable of stimulating the production of cartilage.

13. A method for enhancing cartilage repair in an animal comprising administering to a location where cartilage repair is desired in said animal naked DNA encoding one or more bioactive agent capable of enhancing cartilage repair.

14. A method for the repair and/or regeneration of cartilage in an animal based on the expression of one or more bioactive agent capable of stimulating or otherwise enhancing cartilage regeneration and/or repair, wherein the bioactive agent is expressed from naked DNA encoding the bioactive agent.

15. The method of any of claim 14 or 15 to repair or treat fracture nonunion, large segmental bone or cartilage defects, osteoarthritis, collagen disorders, dwarfism including camptomelic dysplasia, pseudoachondroplasia, multiple epiphyseal dysplasia.

16. The method of any of claims 11-14 wherein the bioactive agent is selected from the group of cartilage morphogens and factors or peptides which block inhibitory signals preventing the repair or regeneration of cartilage.

17. The method of any of claims 11-14 wherein the bioactive agent is a factor selected from the group of cellular growth factors, factors that stimulate chondrogenesis, factors that stimulate migration of stromal cells and factors that stimulate matrix deposition.

18. The method of claim 17 wherein the factor is selected from the group of an FGF, a TGF, and a BMP.

19. The method of claim 18 wherein the factor is BMP-2.

20. A method for enhancing cartilage repair in an animal comprising administering to a location where cartilage repair is desired the composition of any of claims 1-10.


SEQUENCE LISTING (2 sequences)

SEQ ID NO: 1 - DNA, 1547 bp, Homo sapiens
ggggacttcttgaacttgcagggagaataacttgcgcaccccactttgcgccggtgcctttgccccagcggagcctgcttcgccatctccgagccccaccgcccctccactcctcggccttgcccgacactgagacgctgttcccagcgtgaaaagagagactgcgcggccggcacccgggagaaggaggaggcaaagaaaaggaacggacattcggtccttgcgccaggtcctttgaccagagtttttccatgtggacgctctttcaatggacgtgtccccgcgtgcttcttagacggactgcggtctcctaaaggtcgaccatggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggaaaagggcatcctctccacaaaagagaaaaacgtcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgctagtacagcaaaattaaatacataaatatatatata

SEQ ID NO: 2 - protein, 396 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR